(19) United States
(12) Patent Application Publication
(10) Pub. No.: US 2007/0203239 A1
(43) Pub. Date: Aug. 30, 2007

(54) Compounds For The Treatment Of An Acute Injury To The Central Nervous System

(75) Inventors: Josette-Nicole Mahy Gehenne, Alella (ES); Manuel J. Rodriguez Allue, Sant Feliu de Llobregat (ES); Marco Pugliese, Sant Pere de Ribes (ES)

Correspondence Address: TRASK BRITT, P.O. BOX 2550, SALT LAKE CITY, UT, 84110, US

(73) Assignee: Neurotec Pharma, S.L., Barcelona (ES)

(21) Appl. No.: 11/630,420

(22) PCT Filed: Jun. 23, 2005
(86) PCT No.: PCT/ES05/00357

§ 371 Date: Apr. 9, 2007

(30) Foreign Application Priority Data
Jun. 23, 2004 (ES) P200401628

(51) Int. Cl. A61K 31/195

(52) U.S. Cl. 514/561

(57) ABSTRACT

K_{ATP} channel closers (KCCs) are useful for the prophylactic and/or therapeutic treatment of a CNS acute damage in a mammal, including a human, because their administration, particularly in the case of glibenclamide, potientates the neuroprotector microglial effect. Therefore, they may be useful in treating the acute phase of CNS diseases such as stroke, seizure, axonal injury, traumatic damage, neurodegeneration, spinal cord injury, infectious and autoimmune CNS diseases. KCCs, isotopically modified, are also useful for the preparation of diagnostic agents for detection and follow-up of CNS acute damage.

This invention relates to the field of human and animal medicine, and specifically to compounds for the treatment and diagnosis of diseases, in particular, diseases related with the central nervous system acute damage.

### BACKGROUND ART

Microglia are distributed in non-overlapping territories throughout the Central Nervous System (CNS). In functional terms, microglia represents the network of immune accessory cells throughout the brain, spinal cord and eye neurostructures functioning as an intrinsic sensor of threats. The high sensitivity of microglial cells to the CNS microenvironment changes enables them to function as sentinels (cf. G. W. Kreutzberg, Trends Neurosci. 1996, vol. 19, pp. 312-8). Benefits derived from activated microglia remain controversial because of its dual role, protecting the CNS from damage as well as amplifying the effects of inflammation and autoimmune responses and mediating cellular neurodegeneration (cf. W. J. Streit et al., Prog. Neurobiol. 1999, vol. 57, pp. 563-81).

CNS damage rapidly changes neuronal gene expression and stimulates nearby microglia for support. Microglia activation, the first step in the protection of CNS injury (cf. L. Minghetti et al., Prog. Neurobiol. 1998, vol. 54, pp. 99-125) is sufficient to restrain further tissue damage. After an injury, early activated microglial cells secrete anti-inflammatory cytokines (e.g. IL-10 and TGF-beta) and express glutamate transporters to prevent excitotoxic injury.

Thus, in this acute phase, the glutamate clearance by active astrocyte is helped by a de novo microglial Glu transporter expression (cf. A. V. Vallat-Decouvelaere et al., J. Neuropathol. Exp. Neurol. 2003, vol. 62, pp. 475-85) to avoid excitotoxic damage. In this early post-injury event, ramified microglial cell expression of EAAT1, EAAT2 and EAAT3 glutamate transporters prevents glutamate-mediated excitatory neuronal cell death (cf. F. Lopez-Redondo et al., Brain Res. Mol. Brain Res. 2000, vol. 76, pp. 429-35; F. 35 Chretien etal., Neuropathol. Appl. Neurobiol. 2002, vol. 28, pp. 410-7).

At present, very few treatments have been proposed in clinical practice to prevent CNS acute damage. They are orientated to inhibit or prevent activation of mechanisms involved in neuronal death, but their effectiveness is limited and sometimes contradictory. Some agents, such as tirilazad mesylate and Ebselen (2-phenyl-1,2-benzisoselenazol-3(2H)-one) have been proposed to neutralize free radicals and avoid their toxicity; others have been proposed to reduce intracellular calcium toxicity (e.g. nimodipine) or to interfere with GABAergic neurotransmission (e.g. clomethiazol) or with glutamate neurotransmission (e.g. magnesium). However, at present, there is not any treatment to avoid CNS acute injury presenting both a good efficacy and a high security for the patient.

Actually, in most of the times, after an acute damage, patients are kept under clinical observation during some days in absence of a specific treatment just waiting for a beneficial evolution. Thus, it is desirable to provide new therapeutic agents for the early treatment of CNS acute damage.

### SUMMARY OF THE INVENTION

Inventors have surprisingly found that human and rodent activated microglia strongly expresses a K_{ATP} channel similar to the ones known in cardiac and muscular tissues, neurones and pancreatic beta cells. K_{ATP} channels initially found in heart (cf. A. Noma, Nature 1983, vol. 305, pp. 147-8) have also been described in pancreas, skeletal muscle, smooth muscle, pituitary, tubular cells of the kidney, vascular cells and specific neurons of some brain areas.

The fact that activated microglia expresses K_{ATP} channels, turns K_{ATP} channel closers (KCCs) including sulfonylureas, into therapeutic targets to protect CNS from acute damage. KCCs have been used until now for the treatment of diabetes type 2. Inventors have found that the KCCs, and in particular glibenclamide, potentiate acute microglial reaction and avoid AMPA-induced (AMPA: α-amino-3-hydroxy-5-methylisoxazole-4-propionic) brain excitotoxicity in various CNS pathologies such as stroke, seizure, axonal injury, traumatic damage, neurodegeneration, spinal cord injury, infectious and autoimmune diseases. KCCs promote synaptic glutamate removal and anti-inflammatory cytokine secretion by ramified microglia at early survival periods.

Thus, the present invention relates to the use of a KCC, or of an isotopically species modified thereof, for the preparation of a prophylactic, therapeutic and/or diagnostic agent for CNS acute damage in a mammal, including a human. The invention also provides a method of prophylaxis, therapy and/or diagnosis of a mammal, including a human, suffering from or susceptible to CNS acute damage, comprising the administration of an effective amount of a KCC, or of an isotopically modified species thereof, together with appropriate amounts of acceptable diluents or carriers.

KCCs are typically sulfonylureas. Examples of them are glibenclamide, tolbutamide, gliclazide, gliquidone, tolazamide, chlorpropamide, glipizide, glyburide, glimepiride and glisentide. In a particular embodiment of the invention, the KCC is glibenclamide.

In a particular embodiment of the invention, the CNS acute damage is caused by a CNS injury, such as brain injury, spinal cord injury, global ischemia, focal ischemia, hypoxia, stroke, seizure, epilepsy, status epilepticus, the acute phase of CNS vascular disease, neuroophtalmology disease (e.g. inflammation optic neuropathy and retinitis) and trauma. In another embodiment, the CNS acute damage is caused by a CNS degenerative disease. More particularly, the CNS degenerative disease is amyotrophic lateral sclerosis, multiple sclerosis, encephalopathy and adrenoleukodystrophy. In another embodiment, the CNS acute damage is caused by a CNS infectious disease, in particular, by encephalomyelitis and by meningitis caused by viral infection (e.g. HIV encephalitis), parasitic infection (protozoal and metazoal infections), bacterial infection (e.g. purulent leptomeningitis and brain abscess), mycoplasma infection and fungal infection. In another embodiment, the CNS acute damage is caused by an autoimmune disease, particularly, by demyelinating diseases such as multiple sclerosis and phenylketonuria. In another embodiment, the CNS acute damage is caused by a nutritional, metabolic or toxic disorder, in particular by hepatic encephalopathy, lead poisoning and stupefying drug poisoning.

According to the present invention, KCCs prevent CNS acute excitotoxic effects and therefore may be of use in treating the acute phase of CNS diseases.

It will be appreciated that reference to “treatment” is intended to include prophylaxis as well as the alleviation of early symptoms. In this description words “early” and “acute”, as qualifiers of damage, are used with the same meaning.

A person skilled in the art would select an appropriate administration via of KCCs, including glibenclamide, such as oral, buccal, parenteral, depot or rectal administration, or by inhalation or insufflation (either through the mouth or the nose). Oral and parenteral formulations are preferred. Their administration is preferred to be associated with a narrow therapeutic window following the acute damage.

For oral administration, KCCs may take the form of, for example, tablets or capsules prepared by conventional means with pharmaceutically acceptable excipients. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives. Preparations for oral administration may be suitably formulated to give controlled release of the active compound.

Liquid preparations for perioperative CNS surgery including brain, spinal cord and neuroophthalmic procedures may take the form of, for example, solutions or suspensions, or they may be presented as a dry product for its direct application (e.g. powder, gel or impregnated on a solid support) or reconstitution with water or other suitable vehicle (e.g. sterile pyrogen-free water) before use. Such liquid preparations may be prepared by conventional means with pharmaceutically acceptable additives such as emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, and, optionally, multiple active agents (e.g. antibiotics) in a physiological carrier, such as saline or lactated Ringer's solution, as appropriate. The solution is applied by continuous irrigation of a wound during surgical and diagnostic procedures to potentiate neuroprotection of the CNS.

KCCs may be formulated for parental administration by bolus injection or continuous infusion. Formulations for injection may be presented in unit dosage form (e.g. in ampoules or in multidose containers) with an added preservative. The compositions may take forms such as suspensions, solutions or emulsions in oily or aqueous vehicles, and may contain agents such as stabilizing and/or dispersing agents. Alternatively, the active ingredient may be in powder form for constitution with a suitable vehicle (e.g. sterile pyrogen-free water) before use.

KCCs may also be formulated for local administration, for example, by carotid injection, lumbar or cisternal puncture, intracerebroventricular or tissue infusion, as solutions for administration via a suitable delivery device or alternatively as a powder mix with a suitable carrier for administration using a suitable delivery device.

KCCs may also be formulated as rectal compositions such as suppositories or retention enemas (e.g. containing conventional suppository bases such as cocoa buffer or other glycerides).

For intranasal and ocular administration, KCCs may be formulated as solutions for administration via a suitable metered or unit dose device or alternatively as a powder mix with a suitable carrier for administration using a suitable delivery device.

Suitable doses ranges would be routinely found by the person skilled in the art. Thus, for use in conditions according to the present invention, the compounds may be used at doses appropriate for other conditions for which KCCs are known to be useful. It will be appreciated that it may be necessary to make routine variations to the dosage, depending on the age and condition of the patient, and the precise dosage will be ultimately at the discretion of the attendant physician or veterinarian. The dosage will also depend on the route of administration and the particular compound selected. A suitable dose range is for example 0.01 to 1000 mg/kg bodyweight per day, preferably from 0.1 to about 200 mg/kg and more preferably from 0.1 mg/kg to 10 mg/kg,.

The KCCs useful in the present invention may be administered in combination with other KCCs and/or in combination with other therapeutic agents and may be formulated for administration by any convenient route in a convenient manner. Appropriate doses would be routinely found by those skilled in the art.

The invention also refers to the use of an isotopically modified KCC for the preparation of a diagnostic agent for CNS acute damage. The skilled in the art would appropiately choose isotopes and techniques to detect and follow microglial reaction. Functional brain imaging techniques such as positron emission tomography (PET), single-photon emission computed tomography (SPECT) and nuclear magnetic resonance (NMR) may provide an image that represents the distribution in the CNS of the microglial reaction. Once activated, microglia shows a territorially highly restricted involvement in the disease process. This confers to them diagnostic value for the accurate spatial localization of any active disease process. KCCs may be labelled for example with ¹¹C, ¹³C, ¹⁷F, ³¹P, ¹H or ¹⁷O.

Throughout the description and claims the word “comprise” and variations of the word, such as “comprising”, are not intended to exclude other technical features, additives, components, or steps. The abstract of the present application is incorporated herein as reference. Additional objects, advantages and features of the invention will become apparent to those skilled in the art upon examination of the description or may be learned by practice of the invention. The following example and drawings are provided by way of illustration, and are not intended to be limiting of the present invention.

### BRIEF DESCRIPTION OF THE DRAWINGS

FIG. 1 shows the hippocampal microgliosis area (A, in mm²) induced by stereotaxic microinjection of PBS (sham, S), glibenclamide (Glib), AMPA and AMPA+glibenclamide (AMPA+Glib). One asterisk means p<0.01 different from sham and the symbol # means p<0.01 different from AMPA (LSD post-hoc test).

FIG. 2. shows the area (A, in mm²) of hippocampal CA1 lesion induced by stereotaxic microinjection of PBS (sham, S), glibenclamide (Glib), AMPA, or AMPA+glibenclamide (AMPA+Glib). One asterisk means p<0.01 different 35 from sham, and the symbol # means p<0.01 different from AMPA (LSD post-hoc test).

### DETAILED DESCRIPTION OF A PARTICULAR EMBODIMENT

### Glibenclamide Potentiates Microglial Reaction and avoids AMPA Induced Rat Hippocampal Excitotoxic Damage

This model relies on the acute stereotaxic over-activation of rat glutamate hippocampal receptors that results in a neurodegenerative process characterized by a neuronal loss with astroglial and microglial reactions (cf. F. Bernal et al., Hippocampus 2000, vol. 10, pp. 296-304; F. Bernal et al., Exp. Neurol. 2000, vol. 161, pp. 686-95). In this neurodegenerative model, rats were anaesthetized with equithesin (a mixture of chloral hydrate and sodium pentobarbitone; 0.3 ml/100 g body wt, i.p.), and placed on a Kopf stereotaxic frame with the incisor bar set at −3.3 mm. Intracerebral injections aimed at the dorsal hippocampus were performed at 3.3 mm caudal to bregma, 2.2 mm lateral, and 2.9 mm ventral from dura (cf. G. Paxinos et al., “The rat brain in stereotaxic coordinates”, Sydney: Academic Press 1986). A volume of 0.5 μl was injected over a period of 5 min.

Four different groups of rats received two injections in a 2-hour interval as follows: a) sham rats (n=4) received two injections of PBS; b) AMPA rats (n=4) received the first injection of 5.4 mM AMPA and the second of PBS; c) glibenclamide rats (n=4) received two injections of 20 μM glibenclamide; d) AMPA+glibenclamide rats (n=4) received 5.4 mM AMPA+20 μM glibenclamide in the first injection and 20 μM glibenclamide in the second injection. All rats were sacrified 24 hours after the lesion.

Rats were transcardially perfused with 300 ml of 0.1 M phosphate buffer (PB, pH 7.4) followed by 300 ml ice-cold fixative (flow rate 20 ml/min). The fixative consisted of 4% (w/v) paraformaldehyde in PB. Brains were removed, crioprotected with 15% (w/v) sucrose in PB and then, frozen with dry ice.

Cryostat sections (12 μm) were obtained at the level of dorsal hippocampus (−3.3 mm to bregma). Isolectine B4 (IB4) histochemistry was performed to identify the microglial reaction (cf. C. A. Colton et al., J. Histochem. Cytochem. 1992, vol. 40, pp. 505-12). The hippocampal morphology was studied in Cresyl violet stained sections. The area of lesion and the microgliosis evaluation were performed on cresyl violet and IB4-positive stained sections respectively. These parameters were analyzed using a computer-assisted image analysis system (OPTIMAS®, BioScan Inc., Washington, USA). IB4-stained reactive microcytes were counted at ×100 magnification using an ocular grid mounted on a transmission light microscope (Axiolab, Zeiss, Göttingen, Germany). One-way ANOVA was used to compare differences between groups, followed by the LSD post-hoc test. Results are expressed as mean ± SEM. All analyses were performed with the computer program STATGRAPHICS (STSC Inc., Rockville, Md., USA).

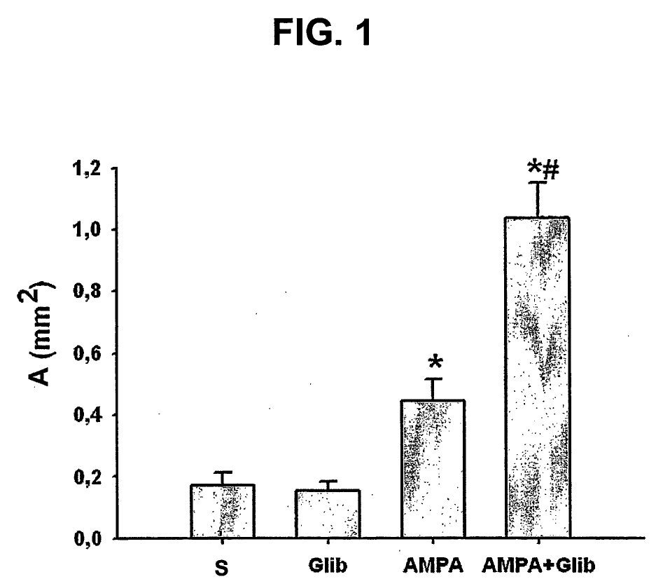

The microglial reaction found in sham and glibenclamide groups was similar, reaching an area of 0.17±0.04 mm² and 0.16±0.03 mm² respectively. In AMPA rats, a strong microgliosis was evidenced with the ameboid microcytes extended through an area of 0.44±0.07 mm². In the AMPA+Glib group this microgliosis area was increased to an area of 1.04±0.11 mm² (236% of the AMPA group) (One-way ANOVA test result: F₃,1₂=31.81; p=0.0001) (cf. FIG. 1).

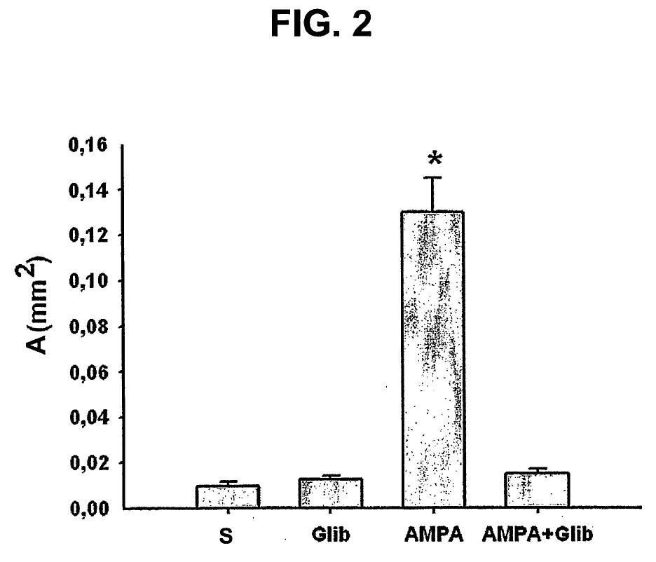

In all four groups the density of reactive microcytes found was similar: 504±82 cells/mm² for sham, 614±91 cells/mm² for glibenclamide, 645±59 cells/mm² for AMPA and 568±56 cells/mm² for AMPA+Glib. As illustrated in FIG. 2 with the quantification of the CA1 pyramidal layer, rich in neuronal cells, in this last group, the 236% increased microglial reaction was associated with an absence of a significant hippocampal lesion. In this layer, the 0.130±0.015 mm² of lesion observed in AMPA rats were decreased to 0.015±0.0016 mm² in the AMPA+Glib group, similar to the areas of 0.009±0.0015 mm² and 0.012±0.0017 mm² found in the sham and glibenclamide groups respectively (One-way ANOVA test result: F₃,11=52.14; p=0.00001).

From these results it is clear that glibenclamide potentiates microglial activation and avoids hippocampal excitotoxic damage. A lack of hippocampal lesion was observed in animals treated with AMPA+glibenclamide in comparison with AMPA treated animals.

1-13. (canceled)

14. Method of prophylaxis, therapy and/or diagnosis of a subject suffering from or susceptible to CNS acute damage, said method comprising:
administering to the subject an effective amount of a K_{ATP} channel closer (KCC), or of an isotopically modified species thereof, together with appropriate amounts of acceptable diluents or carriers.

15. The method according to claim 14, wherein the K_{ATP} channel closer is a sulfonylurea.

16. The method according to claim 15, wherein the damage is caused by a CNS injury.

17. The method according to claim 16, wherein the CNS injury is selected from the group consisting of brain injury, spinal cord injury, global ischemia, focal ischemia, hypoxia, stroke, seizure, epilepsy, status epilepticus, CNS vascular disease, neuroocular disease and trauma.

18. The method according to claim 15, wherein the damage is caused by a CNS degenerative disease.

19. The method according to claim 18, wherein the CNS degenerative disease is selected from the group consisting of amyotrophic lateral sclerosis, multiple sclerosis, encephalopathy and adrenoleukodystrophy.

20. The method according to claim 15, wherein the damage is caused by a CNS infectious disease.

21. The method according to claim 20, wherein the CNS infectious disease is selected from the group consisting of viral infection, parasitic infection, bacterial infection, mycoplasma infection and fungal infection.

22. The method according to claim 15, wherein the damage is caused by an autoimmune disease.

23. The method according to claim 22, wherein the autoimmune disease is selected from the group consisting of multiple sclerosis and phenylketonuria.

24. The method according to claim 15, wherein the damage is caused by a nutritional, metabolic or toxic disorder.

25. The method according to claim 24, wherein the disorder is selected from the group consisting of hepatic encephalopathy, lead poisoning and stupefying drug poisoning.

26. The method according to claim 14, wherein the K_{ATP} channel closer is glibenclamide.

27. The method according to claim 15, wherein the K_{ATP} channel closer is glibenclamide.

28. The method according to claim 16, wherein the K_{ATP} channel closer is glibenclamide.

29. The method according to claim 17, wherein the K_{ATP} channel closer is glibenclamide.

30. The method according to claim 18, wherein the K_{ATP} channel closer is glibenclamide.

31. The method according to claim 19, wherein the K_{ATP} channel closer is glibenclamide.

32. The method according to claim 20, wherein the K_{ATP} channel closer is glibenclamide.
